(19) United States
(12) Patent Application Publication
(10) Pub. No.: US 2008/0300464 A1
(43) Pub. Date: Dec. 4, 2008

(54) Laryngoscope with Timer

(75) Inventors: Atul K. Dhingra, Houston, TX (US); David E. Persse, Kemah, TX (US)

Correspondence Address: ANDREA E. TRAN, 1800 BERING DRIVE , SUITE 540, HOUSTON, TX, 77057, US

(73) Assignee: Atul K. Dhingra, Houston, TX (US)

(21) Appl. No.: 11/754,998

(22) Filed: May 30, 2007

(51) Int. Cl. A61B 1/267

(52) U.S. Cl. 600/199; 600/185

(57) ABSTRACT

A laryngoscope comprising a handle, a blade coupled to the handle, wherein the blade is movable between an operational position and a non-operational position. The laryngoscope also comprises a timer, wherein the timer is in communication with the handle or the blade of the laryngoscope, the timer being operative to measure a time period in which the blade is in the operational position.

### BACKGROUND

1. Technical Field

The present disclosure relates generally to apparatus and methods utilized in medical procedures. In particular, but without limitation, the present disclosure relates to a laryngoscope with a timer and associated methods of operation.

2. Background Information

Patients undergoing general anesthesia, cardiopulmonary resuscitation, or other medical procedures or conditions may require aid in respiration through endotracheal intubation. Endotracheal intubation procedures are commonly employed to secure a controlled airway and to deliver oxygen, gases and other therapeutic agents into the trachea and lungs of patients. The conventional intubation procedure requires a healthcare provider to insert into the oral cavity a laryngoscope with a blade that serves to displace the tongue and allow visualization of the larynx through the mouth. An endotracheal tube is then inserted into place while the laryngoscope blade keeps the patient's airway open. This procedure is done routinely by many types of medical personnel: including but not limited to EMS personnel: ER physicians, Anesthesiologists, Critical Care specialists, and Pulmonologists.

Intubation is a critical lifesaving procedure and if taken too long to perform, may pose risks to the patient. As a patient undergoing tracheal intubation is usually unconscious and in need of respiratory aid, each intubation attempt is a period of time when oxygen is not transported through the patient's body. Particularly, in an emergency setting, an individual rendering the lifesaving procedure is under extreme time constraints. It is recommended by the American Heart Association (AHA) that an intubation attempt should take approximately 30 seconds. Though some hospital settings have monitors to reflect patient status, including blood oxygen level, endotracheal intubation must sometimes be performed without the aid of such monitors. For example, Emergency Medical Technicians (EMTs) sometimes use a laryngoscope to perform tracheal intubation in a prehospital setting. There is currently no mechanism to give a healthcare provider feedback on the duration of intubation.

A laryngoscope comprising the elements of a traditional laryngoscope in communication with a timer may provide immediate feedback to a healthcare provider regarding the duration of intubation. Such device may ensure that an intubation procedure is performed within a recommended period of time. In the case of an unsuccessful intubation attempt, the device alerts the healthcare provider of the period of time that has elapsed. Given this information, the provider will know to oxygenate a patient through alternative means (e.g. bag-valve-mask or BVM) before reattempting the intubation. A laryngoscope in communication with a timer may also be used as a teaching device for medical personnel such as physicians, nurses, medical residents or medical students.

### SUMMARY

The following presents a general summary of some of the many possible aspects of this disclosure in order to provide a basic understanding of this disclosure. This summary is not an extensive overview of all aspects of this disclosure. This summary is not intended to identify key or critical elements of the disclosure or to delineate or otherwise limit the scope of the claims. The following summary merely presents some concepts of the disclosure in a general form as a prelude to the more detailed description that follows.

As described herein, a laryngoscope may comprise a handle and a blade, wherein the blade is movable between an operational position and a non-operational position. The laryngoscope may further include a timer, wherein the timer is in communication with the handle or the blade of the laryngoscope, the timer being operative to measure a time period in which the blade is in the operational position.

The present disclosure further describes a method of operating a laryngoscope including moving a blade into an operational position relative to a handle of the laryngoscope. The method may further include activating a timer, wherein the timer is in communication with the laryngoscope, the timer being operative to measure a time period in which the blade is in the operational position.

### BRIEF DESCRIPTION OF THE DRAWINGS

The following drawings illustrate some of the many possible aspects, embodiments and/or implementations of this disclosure in order to provide a basic understanding of this disclosure. These drawings do not provide an extensive overview of all aspects, embodiments or implementations of this disclosure. These drawings are not intended to identify key or critical elements of the disclosure or to delineate or otherwise limit the scope of the claims. It should be understood that the disclosure is not limited to the precise arrangements and instrumentalities shown in the drawings. The following drawings merely present some concepts of the disclosure in a general form. Thus, for a detailed understanding of this disclosure, reference should be made to the following detailed description, taken in conjunction with the accompanying drawings, in which like elements have been given like numerals.

FIG. 1 is an isometric view of a laryngoscope according to the present disclosure.

FIGS. 2 & 4 are alternative views of a laryngoscope according to the present disclosure.

FIG. 3 depicts an illustrative implementation of a laryngoscope with a visual indication according to the present disclosure.

FIG. 5 depicts an illustrative implementation of a laryngoscope in a non-operational mode.

### DETAILED DESCRIPTION

Before the present apparatus and methods are described, it is to be understood that this disclosure is not limited to the particular apparatus or methodology described, as such methods or apparatus may vary. One of ordinary skill in the art should understand that the terminology used herein is for the purpose of describing possible aspects, embodiments and/or implementations only, and is not intended to limit the scope of the present disclosure which will be limited only by the appended claims.

It must also be noted that as used herein and in the appended claims, the singular forms “a,” “and,” and “the” may include plural referents unless the context clearly dictates otherwise. Thus, for example, reference to “a device” refers to one or several devices, and reference to “the method of operating” includes reference to equivalent steps and methods known to those skilled in the art, and so forth.

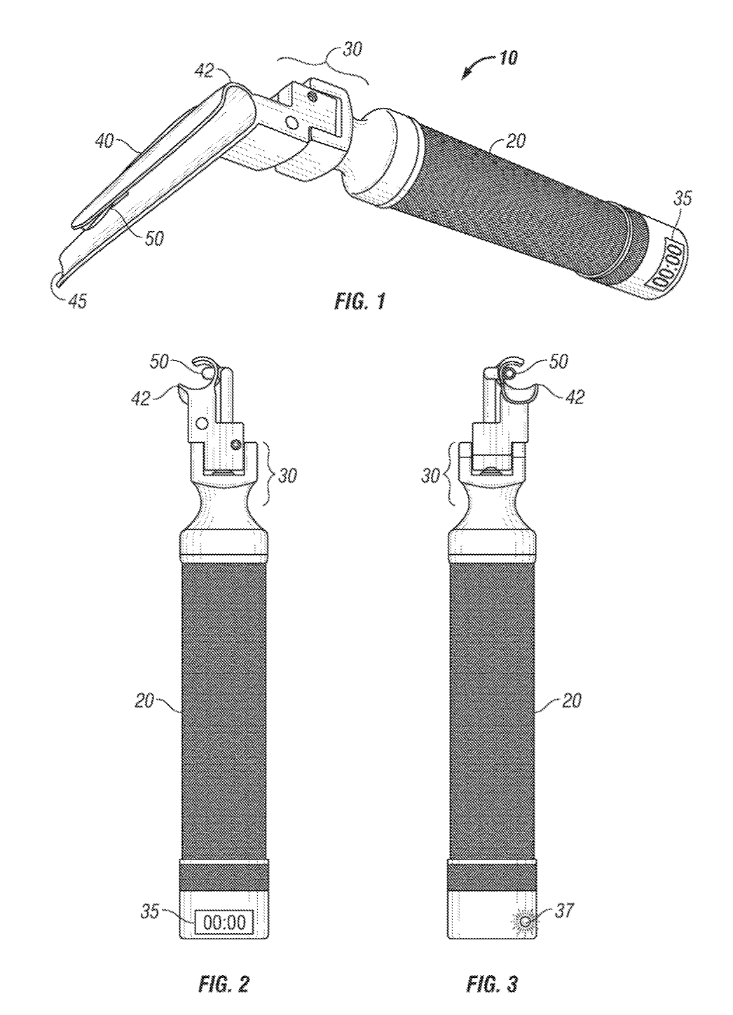

FIGS. 1, 2 & 4 show various views of a laryngoscope 10 according to the present disclosure. It should be understood that this disclosure has applicability to laryngoscopes in general and is not intended to be limited as specifically described. As seen in FIGS. 1, 2 4, a laryngoscope 10 may comprise a handle 20, a blade 40, a light source 50 and a timer 35. The components of a laryngoscope are substantially rigid and may be constructed of materials including but not limited to metal or metal alloys, stainless steel, polymers, disposable material, or any suitable material as known to one skilled in the art.

As shown in FIGS. 1, 2 & 4, the blade 40 has a distal end 45, i.e., blade tip, which is the end that is first inserted into the cavity, and a proximal end 42 which is coupled to the handle 20. As used here, the blade 40 may also refer to, but is not limited to a spatula or a plate. It is contemplated that the blade 40 may take various forms and in some implementations may be substantially straight, curved, or substantially straight with a curved tip. The interior surface of the blade 40 may create a curved cavity, either concave or convex, that facilitates the insertion of an endotracheal tube while the outer surface may contact the tongue of a patient during an intubation procedure. In one possible implementation, the cross-section of the blade 40 may be substantially circular.

As shown in FIGS. 1, 2 & 4, the handle 20, which may have a generally cylindrical form, can be coupled to the blade 40 in a substantially L-shaped configuration. The handle 20 may also serve as either a surface for gripping the laryngoscope 10 or as a housing for a hollow body or a power supply as to be described below.

It is contemplated that the blade 40 may be coupled to the handle 20 in various ways. In one aspect, the blade 40 may be coupled directly to the handle 20. In one possible implementation, the handle 20 is detachably or removably coupled to the blade 40. In another illustrative implementation, the blade 40 may be pivotably coupled to the handle 20, wherein the blade 40 pivots between an operational position and a non-operational position. The laryngoscope 10 may be in an operational position, i.e. locked position when the blade 40 is in a substantially 90 degree angle with the handle 20, or when the opening of the angle between the blade 40 and the handle 20 come to a stopping point. This operational position also describes a position of the blade 40 relative to the handle 20 that is the intended position for the laryngoscope 10 to be used to facilitate tracheal intubation. In an implementation, a laryngoscope 10 is in the operational position when the light source 50 is activated. Conversely, a non-operational position describes a position where the blade 40 is not in a substantially 90 degree angle with the handle 20 and the blade 40 is not independently in a fixed position. In an illustrative implementation, a laryngoscope 20 is in a non-operational position when the light source 50 is deactivated. In another aspect, the handle 20 and the blade 40 may be integrally formed of a single piece of material.

In yet another aspect shown in FIGS. 1, 2 & 4, the blade 40 may be coupled to the handle 20 via a connecting member 30. The connecting member 30 may comprise a groove or slot to allow insertion of the base of the blade 40. Any suitable means for coupling the blade 40 to the handle 20 may be provided as is known in the art. It is understood that the connecting member 30 may be a separate element from the handle 20. Alternatively, the connecting member 40 and the handle 20 may be integrally formed of a single piece of material in which case the connecting member 30 may serve as a mere extension of the handle 20, at the point of coupling to the blade 40.

Still referring to FIGS. 1, 2 & 4, the timer 35 may be in communication with the blade 40 and the handle 20. In illustrative implementations, the timer 35 may be in wireless communication with the laryngoscope 10 via signals including, but not limited to, RF and Bluetooth. As shown, the timer 35 can be coupled to the handle 20 but the present disclosure contemplates that the timer 35 may be coupled to any part of the laryngoscope 10 such as the connecting member 30, the blade 40 or the like. The timer 35 is an apparatus which operates to measure any time period and in particular, it may measure the time in which the blade 40 is in an operational position relative to the handle 20. In one implementation, the timer 35 may be automatically activated when the blade 40 is moved to an operational position. In another illustrative implementation, the timer 35 may be automatically activated once the light source 50 is activated. In yet another non-limiting implementation, the timer 35 may be reset to zero whenever the blade 40 is moved to a non-operational position, thus the timer 35 only measures time periods in which the blade 40 is in a continuously operational position. In yet another implementation, the timer 35 may be activated manually by the user.

The timer 35 may also provide an indication, audio and/or visual in nature of the time period measured for intubation. An audio indication may include any audible sound, such as, but not limited to, a beep, buzz, alarm or the like. A visual indication may include, but is not limited to, a light or a digital display, analog display of the measured time period or the like.

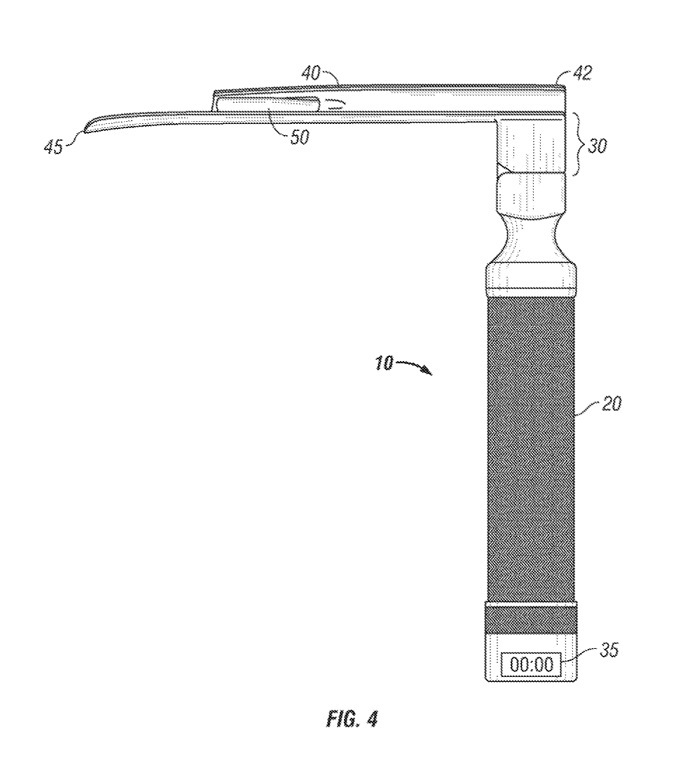

As shown in FIG. 3, one aspect of the present disclosure is a laryngoscope 10 which comprises a handle 20 coupled to a proximal end 42 of a blade via a connecting member 30, wherein a visual indication 37, e.g., blinking light, is coupled to the handle 20. Also shown in FIG. 3 is a light source 50 disposed within the blade.

The indication, which may be visual, audio and/or the like, may occur at the conclusion of different time periods. In several aspects, the time period could begin automatically at the time that the laryngoscope blade 40 is moved into an operational position from a non-operational position or the time period may be manually set by a user. In one possible implementation, a predetermined time period may be set or programmed by a user and the timer provides an indication when the time period has elapsed. By way of example, a user may program the laryngoscope timer 35 to give an audio, visual or other indication following the conclusion of 30 seconds as recommended by the AHA for proper intubation time. In another illustrative implementation, a user may program the laryngoscope 10 to provide an indication following the conclusion of a set interval of time. By way of example, a user may program the laryngoscope timer 35 to give an audio, visual or other indication after every 15 second interval. Alternatively, the laryngoscope timer 35 may provide an audio, visual or other indication after 30 seconds, another indication after 45 seconds, followed by a constant indication after 1 minute. In yet another non-limiting implementation, the time period may be adjustable by the user according to the user's own setting preferences, or according to specific needs of the patient undergoing tracheal intubation.

As depicted in FIGS. 1, 2 & 4, the laryngoscope 10 may also comprise a light source 50. As shown, the light source 50 or light-emitting element may be positioned within the interior curve of the blade 40 for communicating light to the area adjacent the distal end of the blade 45. During the operation of the laryngoscope 10, the light source 50 may provide illumination to the cavity or orifice of the patient. It should be understood that the light source 50 may be positioned on or coupled to any element of the laryngoscope 10 to provide illumination to the area near the distal end of the blade 45 or the cavity of a patient. The light source 50 may be activated automatically once the blade 40 is moved into an operational position. The light source 50 may also be activated manually, based on the user's preference or specification. In one possible implementation, the light source 50 may include a light bulb to illuminate the distal end of the blade 45.

A power supply (not shown) for the light source 50 may be housed within the handle 20. In illustrative implementations, the power supply may be coupled to any element of the laryngoscope 10, including but not limited to the handle 20 the blade 40, or the connecting member 30. The power supply may comprise a battery, a chargeable power source, any circuitry or the like to power the laryngoscope 10.

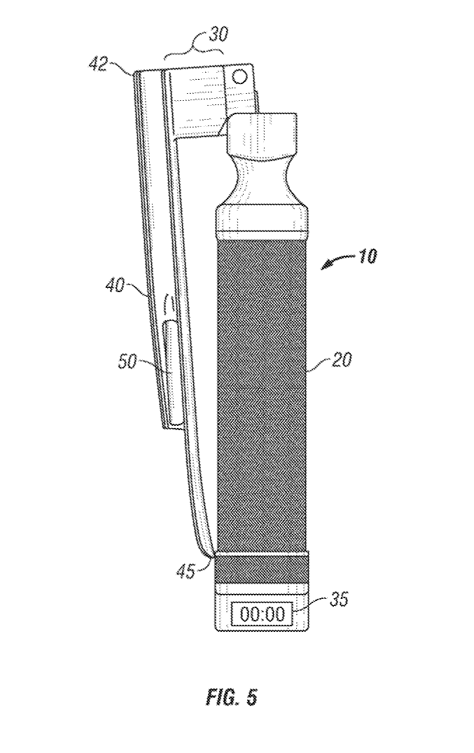

Referring now to FIG. 5, the laryngoscope 10 is depicted, according to one implementation, with its blade 40 in a non-operational position relative to the handle 20. When the blade 40 is in a non-operational position as shown, the light source 50 and/or the timer 35 may de-activated. It is contemplated that a non-operational position of the blade 40 may be as shown in FIG. 5 or in any suitable configuration where the blade 40 is not in an operational position as described herein.

The present disclosure also describes methods for operating a laryngoscope. The present disclosure contemplates various methods comprising all or less than all of the steps described herein, any number of repeats of any of the steps described herein, and in any order. One of skill in the art will appreciate that the laryngoscope of the present method may include all elements as previously described herein. A method may include moving a laryngoscope blade into an operational position relative to a handle of the laryngoscope. The method may also include activating a timer, wherein the timer is in communication with the laryngoscope, the timer being operative to measure a time period in which the blade is in the operational position. Furthermore, the method may include the step of setting an amount of time, wherein the timer provides a visual and/or audio indication of when the amount of time has elapsed.

The activating of the timer may be by any suitable means including but not limited to by user specification or by automatic means. According to one implementation, a method for operating a laryngoscope may include automatically activating a timer when the laryngoscope blade is moved into an operational position. In another implementation, a method for operating a laryngoscope may include resetting the timer to zero when the blade is moved to a non-operational position.

The methods of operating a laryngoscope may also include the step of setting particular periods of time on the timer. According to one implementation, a method of operating a laryngoscope may include the step of setting a predetermined amount of time on the timer wherein the timer provides an audio, visual or other indication when the predetermined time has elapsed on the timer. By way of example, a user may operate a laryngoscope by setting a particular time period on the timer, such as but not limited to, 30 seconds wherein the timer triggers a beep following the conclusion of the seconds to provide feedback to the healthcare provider regarding the length of intubation time. According to yet another implementation, a method of operating a laryngoscope may include the step of setting an interval or duration of time on the timer wherein the timer provides an audio, visual or other indication when the interval of time has elapsed on the timer. As another non-limiting example, a user may operate a laryngoscope by setting 15 second intervals on the timer to enable the timer to generate a sound or blinking light each time 15 seconds has elapsed on the timers thus giving an indication of the ongoing duration of intubation time.

A person of skill in the art would appreciate, upon consideration of the above disclosure and accompanying drawings, that numerous modifications, additions, substitutions, variations, deletions and other changes may be made without departing in any way from the spirit or scope of the present disclosure and/or claims below. The embodiments, implementations and/or aspects of the present disclosure are, therefore, to be considered in all respects as illustrative and not restrictive.

What is claimed is:

1. A laryngoscope comprising:
a handle;
a blade coupled to the handle, wherein the blade is movable between an operational position and a non-operational position; and
a timers wherein the timer is in communication with the handle or the blade of the laryngoscope, the timer being operative to measure a time period in which the blade is in the operational position.

2. The laryngoscope of claim 1, wherein the timer is coupled to the handle or the blade of the laryngoscope.

3. The laryngoscope of claim 1, wherein the timer is in wireless communication with the handle or the blade of the laryngoscope.

4. The laryngoscope of claim 3, wherein the timer is in wireless communication via RF signals.

5. The laryngoscope of claim 3, wherein the timer is in wireless communication via Bluetooth signals.

6. The laryngoscope of claim 1, wherein the timer is reset to zero when the blade is in the non-operational position.

7. The laryngoscope of claim 1, wherein the timer is automatically activated when the blade is moved into the operational position.

8. The laryngoscope of claim 1, wherein the timer is activated by a user.

9. The laryngoscope of claim 1, wherein the timer provides a visual indication of the time period measured.

10. The laryngoscope of claim 9, wherein the timer allows a user to set a predetermined amount of time and the timer provides the visual indication of when the predetermined amount of time has elapsed.

11. The laryngoscope of claim 9, wherein the timer allows a user to set an interval of time and the timer provides the visual indication for each instance that the interval of time has elapsed.

12. The laryngoscope of claim 1, wherein the timer provides an audio indication of the time period measured.

13. The laryngoscope of claim 12, wherein the timer allows a user to set a predetermined amount of time and the timer provides the audio indication of when the predetermined amount of time has elapsed.

14. The laryngoscope of claim 12, wherein the timer allows a user to set an interval of time and the timer provides the audio indication for each instance that the interval of time has elapsed.

15. The laryngoscope of claim 1, wherein the blade is detachably coupled to the handle.

16. The laryngoscope of claim 1, wherein the blade is coupled to the handle via a connecting member.

17. The laryngoscope of claim 1, wherein the blade is pivotably coupled to the handle.

16. The laryngoscope of claim 1, further comprising a light source positioned upon the blade or the handle.

17. The laryngoscope of claim 1, wherein the handle comprises a hollow body.

18. The laryngoscope of claim 17, wherein a power supply for powering the timer is positioned within the body.

19. The laryngoscope of claim 18, wherein the power supply comprises at least one battery.

20. A method of operating a laryngoscope, the method comprising:
moving a blade into an operational position relative to a handle of the laryngoscope; and
activating a timer, wherein the timer is in communication with the laryngoscope, the timer being operative to measure a time period in which the blade is in the operational position.

21. The method of claim 20, wherein the timer is coupled to the laryngoscope.

22. The method of claim 20, wherein the timer is in wireless communication with the laryngoscope.

23. The method of claim 22, wherein the timer is in wireless communication via RF signals.

24. The method of claim 22, wherein the timer is in wireless communication via Bluetooth signals.

25. The method of claim 20, wherein the timer is reset to zero when the blade is in the non-operational position.

26. The method of claim 20, wherein the timer is automatically activated when the blade is moved into the operational position.

27. The method of claim 20, wherein the timer is activated by a user.

28. The method of claim 20, wherein the timer provides a visual indication of the time period measured.

29. The method of claim 28 further comprising setting a predetermined amount of time on the timer wherein the timer provides the visual indication of when the predetermined amount of time has elapsed.

30. The method of claim 28 further comprising setting an interval of time on the timer, wherein the timer provides the visual indication for each instance that the interval of time has elapsed.

31. The method of claim 20, wherein the timer provides an audio indication of the time period measured.

32. The method of claim 20 further comprising setting a predetermined amount of time on the timer, wherein the timer provides the visual indication of when the predetermined amount of time has elapsed.

33. The method of claim 20 further comprising setting an interval of time on the timer, wherein the timer provides the visual indication for each instance that the interval of time has elapsed.

34. The method of claim 20, wherein the blade is detachably coupled to the handle.

35. The method of claim 20, wherein the blade is coupled to the handle via a connecting member.

36. The method of claim 20, wherein the blade is pivotably coupled to the handle.

37. The method of claim 20, wherein the laryngoscope further comprises a light source positioned upon the blade or the handle.

38. The method of claim 20, wherein the handle comprises a hollow body.

39. The method of claim 38, wherein a power supply for powering the timer is positioned within the body.

40. The method of claim 39, wherein the power supply comprises at least one battery.
